(19) United States
(12) Patent Application Publication
(10) Pub. No.: US 2026/0028663 A1
(43) Pub. Date: Jan. 29, 2026

(54) Method And Device For Nucleic Acid Extraction And Detection

(71) Applicant: Yocon Biology Technology Company, Beijing (CN)

(72) Inventors: Shaokun Wang, Beijing (CN); Pandeng Yan, Beijing (CN); Zhen Chen, Beijing (CN)

(21) Appl. No.: 18/996,471

(22) PCT Filed: Jul. 14, 2023
(86) PCT No.: PCT/CN2023/107393

§ 371 Date: Jan. 17, 2025

(30) Foreign Application Priority Data
Jul. 22, 2022 (CN) 202210874566.1

(51) Int. Cl. C12Q 1/6806; C12N 15/10

(52) U.S. Cl. CPC C12Q 1/6806; C12N 15/1006

(57) ABSTRACT

A nucleic acid extraction element is provided, which is made of one or more of polyacrylate, polyamide, polyester, polyurethane, and polylactic acid (PLA), where the extraction element adsorbs nucleic acid in a first solution with a pH value of 2 to 6, and releases the nucleic acid in a second solution with a pH value of 8 to 12. Therefore, the extraction element can be used for extraction and detection of the nucleic acid, greatly shorten the time for extraction, cleaning, and purification of the nucleic acid, simplify operating steps for extraction and purification of the nucleic acid, and reduce consumption of equipment for extraction and purification of the nucleic acid.

### CROSS-REFERENCE TO RELATED APPLICATION

The present application is a national stage application of International Patent Application No: PCT/CN2023/107393, filed on Jul. 14, 2023 and claims priority to the Chinese Patent Application No. 202210874566.1, filed with the China National Intellectual Property Administration (CNIPA) on Jul. 22, 2022 and entitled “METHOD AND DETECTION DEVICE FOR NUCLEIC ACID EXTRACTION”, which is incorporated herein by reference in its entirety.

### TECHNICAL FIELD

The present disclosure relates to the technical field of molecular biology, and in particular to a method and a device for extracting nucleic acid.

### BACKGROUND

In recent years, molecular biological diagnostic technology based on nucleic acid detection has attracted increasing attention. Nucleic acid is the basis of biological detection in technologies such as polymerase chain reaction (PCR), and improving the efficiency of nucleic acid extraction and shortening a duration of nucleic acid extraction are the focus of researchers.

Among the current nucleic acid extraction methods, the classic phenol-chloroform extraction method requires highly toxic organic solvents such as phenol-chloroform to allow extraction, has complex operations and unstable extraction results, and thus is rarely used in clinical genetic testing. The manual column method has a low missed detection rate and high sensitivity, but is time-consuming, and especially shows a greatly increased time cost when there is a high sample volume, thereby being not suitable for screening large batches of samples. The one-step method (i.e., directly releasing the nucleic acid in a sample to be tested using a sample releaser) has simple steps but a high missed detection rate, and is not suitable for clinical use. The magnetic bead method has a high purity of extracted nucleic acid, can be used in combination with an automatic nucleic acid extractor, has high extraction efficiency, and is suitable for the extraction of large quantities of samples. However, a nucleic acid extraction kit for the magnetic bead method has a complicated extraction procedure, requires an external magnetic field, and takes at least 20 min in the laboratory to obtain the target nucleic acid, which makes it difficult to meet the requirement for rapid nucleic acid detection. Moreover, magnetic beads need to be stored at 4° C., while a large number of waste pipette tips may be generated during the extraction. Therefore, there is an urgent need to develop a nucleic acid extraction and detection method and device that are rapid, easy to use, and low in cost.

### SUMMARY

The present disclosure provides a method for nucleic acid extraction and detection and related products thereof to solve the technical problems of the existing magnetic bead method, such as complicated operation steps, long time, high temperature requirement, external magnetic field demand, and waste of consumables.

A current process of nucleic acid extraction by the magnetic bead method is generally as follows: sampling→sending an obtained sample to the laboratory to allow nucleic acid extraction and purification using a nucleic acid extraction kit→conducting nucleic acid detection (FIG. 1). The steps of nucleic acid extraction and purification in the laboratory are cumbersome and require lysis, adsorption, washing, and elution. Detection of a single sample takes at least 20 min, and the simultaneous operation of multiple samples takes even longer. The nucleic acid extraction method provided by the present disclosure includes: starting nucleic acid extraction immediately after sampling→sending an obtained sample to the laboratory to allow nucleic acid purification→conducting nucleic acid detection (FIG. 2). This “immediate collection and extraction” method makes full use of duration from the sampling to the sample delivery to the laboratory (generally at least 20 min), such that nucleic acid purification can be started when the sample arrives at the laboratory, and takes only about 4 min. The entire process does not require low temperature, external magnetic field, or other consumables such as pipette tips, and no reagents are added; moreover, the nucleic acid yield is comparable to that of the mainstream product magnetic bead method currently on the market.

The present disclosure provides a nucleic acid extraction element, including one or more of polyacrylate, polyamide, polyester, polyurethane, and polylactic acid (PLA), where the extraction element adsorbs the nucleic acid in a first solution with a pH value of 2 to 6, preferably 3 to 4, and releases the nucleic acid in a second solution with a pH value of 8 to 12, preferably 10 to 11; and wherein the nucleic acid includes DNA and/or RNA and/or a fragment thereof.

Those skilled in the art will appreciate that various polyacrylates, polyamides, polyesters, polyurethanes, and PLAs can be used in the present disclosure. In a preferred embodiment, the polyacrylate is, for example, polymethyl methacrylate (PMMA); the polyester is, for example, polyethylene terephthalate (PET).

Those skilled in the art will appreciate that the nucleic acid extraction element in the present disclosure may be in any shape, including a regular or irregular shape, such as a sheet, a layer, a sphere, an ellipsoid, a cylinder, a truncated cone, a cone, a pyramid, a prism, a prism-shaped pyramid, a cube, and a cuboid.

In the present disclosure, the nucleic acid extraction element can be used to extract and purify the nucleic acid, and the extracted and purified nucleic acid can be further used for nucleic acid detection. Those skilled in the art will appreciate that the nucleic acid extraction element can be used for nucleic acid detection of microorganisms (such as viruses, bacteria, fungi, actinomycetes, rickettsia, mycoplasma, chlamydia, and spirochetes) in the human body, animal body, or environment (such as on the surface of objects or food, in the soil, and in the air), and can also be used for the detection of disease-related genes (such as cancer-related genes, hereditary disease-related genes) in the human body or animal body.

Those skilled in the art can determine the volume and the surface area of the nucleic acid extraction element according to specific application of the nucleic acid extraction element, such as the amount of nucleic acid to be extracted and the amount of nucleic acid required for nucleic acid detection. For example, the nucleic acid extraction element has a volume of (0.1-2) cm³, preferably (0.3-1) cm³, and more preferably (0.4-0.6) cm³; for example, the nucleic acid extraction element has a surface area of not less than 5 cm², preferably (10-25) cm², and more preferably (13-16) cm².

Those skilled in the art will appreciate that the nucleic acid extraction element can be prepared by methods known in the art. For example, fibers (such as monofilament fibers) can be woven, needled, or wound into single-layer or multi-layer fabrics (such as mesh fabrics, non-woven fabrics), spheres, ellipsoids, cylinders, truncated cones, cones, pyramids, prism-shaped pyramids, prisms, cubes, and cuboids. Alternatively, fibers (such as monofilament fibers) can be woven, needled, or wound into single-layer or multi-layer mesh fabrics, and then further made into sheets, layers, spheres, ellipsoids, cylinders, truncated cones, cones, pyramids, prisms, prism-shaped pyramids, cubes, and cuboids by welding (such as heating and melting). Alternatively, porous sheets, layers, spheres, ellipsoids, cylinders, truncated cones, cones, pyramids, prisms, prism-shaped pyramids, cubes, and cuboids can be made by a process such as foaming.

Those skilled in the art can understand that the nucleic acid extraction element can exist and be used alone, or can be fixed on a rod-shaped support by winding and/or welding (such as melting at elevated temperature) and the like for use. The extraction element can wrap the entire rod-shaped support, or can wrap part of the rod-shaped support; for example, it is arranged at or wrapped on one end of the rod-shaped support, or fixed to one end of the rod-shaped support by welding (such as melting at elevated temperature) with short fibers to form a brush shape.

Preferably, the nucleic acid extraction element is arranged at or wrapped on one end of a rod-shaped support. For example, fibers (such as monofilament fibers) are fixed to one end of the rod-shaped support by winding and/or welding (such as melting by heating); alternatively, fibers (such as monofilament fibers) are first woven, needle-punched, or wound into a single-layer or multi-layer fabric, and then fixed to one end of the rod-shaped support by winding and/or welding (such as melting by heating). Preferably, the nucleic acid extraction element has a length of 1 cm to 2 cm in the direction along the rod-shaped support; and preferably, the fibers have a diameter of 0.05 mm to 0.5 mm, preferably 0.1 mm to 0.3 mm. Preferably, the rod-shaped support has a breakable point at one end close to the nucleic acid extraction element.

The present disclosure further provides a method for extracting nucleic acid, including using the nucleic acid extraction element.

The present disclosure further provides a method for detecting nucleic acid, including using the nucleic acid extraction element.

Those skilled in the art will appreciate that the extraction method and the nucleic acid detection method may not be aimed at diagnosing a disease.

Preferably, the extraction method or the detection method includes the following steps: placing a sample to be tested in a first solution with a pH value of 2 to 6, preferably 3 to 4, such that the nucleic acid extraction element adsorbs the nucleic acid; and allowing the nucleic acid extraction element to release the nucleic acid in a second solution with a pH value of 8 to 12, preferably 10 to 11.

Preferably, the extraction method or the detection method includes the following steps: placing the sample to be tested in a first solution with a pH value of 2 to 6, preferably 3 to 4, such that the nucleic acid extraction element adsorbs the nucleic acid; washing the nucleic acid extraction element in a third solution with a pH value of 3 to 6, preferably 5 to 6; and allowing the nucleic acid extraction element to release the nucleic acid in a second solution with a pH value of 8 to 12, preferably 10 to 11.

In some embodiments of the present disclosure, the first solution can be referred to as an extraction solution, in which the nucleic acid extraction element can adsorb the nucleic acid; in a preferred embodiment, in the extraction solution, a biological membrane such as a cell membrane is lysed such that a nucleic acid to be extracted or detected is freed into the extraction solution and then adsorbed by the nucleic acid extraction element. The third solution can be referred to as a cleaning solution, which is used to clean the nucleic acid extraction element to remove impurities such as sugars, proteins, and lipids. The second solution can be referred to as a purification solution, which releases the nucleic acid adsorbed on the nucleic acid extraction element.

Preferably, the first solution is a nucleic acid lysis buffer; those skilled in the art will appreciate that a pH regulator may be added to adjust a pH value of the nucleic acid lysis buffer to 2 to 6, preferably 3 to 4. Those skilled in the art will appreciate that when a nucleic acid to be extracted or detected is in a biological membrane such as a cell membrane, the nucleic acid lysis buffer can lyse the biological membrane such as the cell membrane to free the nucleic acid to be extracted or detected into the solution. Those skilled in the art will appreciate that any nucleic acid lysis buffer commonly used in the art can be used in the present disclosure.

Preferably, the nucleic acid lysis buffer includes 0.5 M to 1.5 M of arginine, 0.2 M to 1.2 M of inorganic salt, and a first buffer, and has a pH value of 2 to 6, preferably 3 to 4;
preferably, arginine has a concentration of 0.6 M to 1.2 M;
preferably, the inorganic salt has a concentration of 0.5 M to 1 M;
preferably, the inorganic salt is selected from the group consisting of sodium chloride, potassium chloride, sodium citrate, and a mixture of any two or more thereof;
preferably, the first buffer has a concentration of 5 mM to 30 mM, more preferably 10 mM to 25 mM;
preferably, the first buffer is selected from the group consisting of the MES buffer, the Tris buffer, the acetic acid-sodium acetate buffer, and the citric acid-sodium citrate buffer;
preferably, the nucleic acid lysis buffer further includes a pH regulator.

Preferably, the nucleic acid lysis buffer further includes a metal chelator; preferably, the metal chelator includes EDTA and/or a salt of EDTA including disodium EDTA, tetrasodium EDTA, and calcium disodium EDTA; preferably, the metal chelator has a concentration of 1 mM to 20 mM, more preferably 5 mM to 10 mM;
preferably, the nucleic acid lysis buffer consists of the components defined as above.

In the present disclosure, the nucleic acid lysis buffer does not contain surfactants and guanidine salts, thereby reducing the risk of sample contamination and avoiding the risk of guanidine salts to precipitate at lower temperatures.

Preferably, the second solution comprises a second buffer; those skilled in the art will appreciate that a pH regulator may be added to adjust a pH value of the second buffer to 8 to 12, preferably 10 to 11. Preferably, the second buffer is selected from the group consisting of a DPBS buffer, a Tris buffer, a glycine-sodium hydroxide buffer, and a disodium hydrogen phosphate-sodium hydroxide buffer. Preferably, the Tris buffer, the glycine-sodium hydroxide buffer, and the disodium hydrogen phosphate-sodium hydroxide buffer each have a concentration of 1 mM to 30 mM, preferably 5 mM to 20 mM.

Preferably, the third solution comprises a third buffer; those skilled in the art will appreciate that a pH regulator may be added to adjust a pH value of the third buffer to 3 to 6, preferably 5 to 6. Preferably, the third buffer is selected from the group consisting of a Tris buffer, an MES buffer, an acetic acid-sodium acetate buffer, and a citric acid-sodium citrate buffer. Preferably, the third buffer has a concentration of 1 mM to 20 mM, more preferably 5 mM to 15 mM; preferably, the third buffer further includes a metal chelator; preferably, the metal chelator includes EDTA and/or a salt of EDTA including disodium EDTA, tetrasodium EDTA, and calcium disodium EDTA; preferably, the metal chelator has a concentration of 0.5 mM to 10 mM, more preferably 1 mM to 5 mM.

Those skilled in the art will appreciate that any pH regulator commonly used in the art can be used in the present disclosure, such as an aqueous sodium hydroxide solution, an aqueous ammonia solution, and an aqueous hydrochloric acid solution.

According to the present disclosure, adsorption, washing, and release of the nucleic acid can be done at not less than 0° C. to 70° C., preferably 4° C. to 60° C., and more preferably at a room temperature.

Generally speaking, the adsorption of the nucleic acid can be completed in only 10 min and does not affect the detection results for up to 1 week. A container containing the first solution may be allowed to stand, shaken, or vortexed to complete the adsorption of the nucleic acid.

Generally speaking, the cleaning of the nucleic acid extraction element only takes 20 s, preferably 30 s. Accordingly, the nucleic acid extraction element is cleaned for at least 20 s, preferably at least 30 s, and more preferably 30 s to 2 min. A container containing the third solution may be allowed to stand, shaken, or vortexed to complete the cleaning.

Generally speaking, the release of the nucleic acid takes only 2 min, preferably 3 min. Accordingly, the nucleic acid is released for at least 2 min, preferably at least 3 min, and more preferably 3 min to 5 min. A container containing the second solution may be allowed to stand, shaken, or vortexed to complete of the release of the nucleic acid.

The present disclosure further provides a kit or set for nucleic acid extraction or detection, including the nucleic acid extraction element.

Preferably, the kit or set further includes a first solution with a pH value of 2 to 6, preferably 3 to 4 that is placed in a first container. More preferably, the kit or set further includes a second solution with a pH value of 8 to 12, preferably 10 to 11 that is placed in a second container. More preferably, the kit or set further includes a third solution with a pH value of 3 to 6, preferably 5 to 6 that is placed in a third container. In some embodiments of the present disclosure, the first container can be referred to as a sampling tube, and the first solution can be referred to as an extraction solution, in which the nucleic acid extraction element can adsorb the nucleic acid; in a preferred embodiment, in the extraction solution, a biological membrane such as a cell membrane is lysed such that a nucleic acid to be extracted or detected is freed into the extraction solution and then adsorbed by the nucleic acid extraction element. The third container can be referred to as a cleaning tube, and the third solution can be referred to as a cleaning solution, which is used to clean the nucleic acid extraction element to remove impurities. The second container can be referred to as a purification tube, and the second solution can be referred to as a purification solution, which releases the nucleic acid adsorbed on the nucleic acid extraction element.
preferably, the first solution is a nucleic acid lysis buffer; those skilled in the art will appreciate that a pH regulator may be added to adjust a pH value of the nucleic acid lysis buffer to 2 to 6, preferably 3 to 4.

Preferably, the nucleic acid lysis buffer includes 0.5 M to 1.5 M of arginine, 0.2 M to 1.2 M of inorganic salt, and a first buffer, and has a pH value of 2 to 6, preferably 3 to 4;
preferably, arginine has a concentration of 0.6 M to 1.2 M;
preferably, the inorganic salt has a concentration of 0.5 M to 1 M;
preferably, the inorganic salt is selected from the group consisting of sodium chloride, potassium chloride, sodium citrate, and a mixture of any two or more thereof;
preferably, the first buffer has a concentration of 5 mM to 30 mM, more preferably 10 mM to 25 mM;
preferably, the first buffer is selected from the group consisting of an MES buffer, a Tris buffer, an acetic acid-sodium acetate buffer, and a citric acid-sodium citrate buffer;
preferably, the nucleic acid lysis buffer further includes a pH regulator.

Preferably, the nucleic acid lysis buffer further includes a metal chelator; preferably, the metal chelator includes EDTA and/or a salt of EDTA including disodium EDTA, tetrasodium EDTA, and calcium disodium EDTA; preferably, the metal chelator has a concentration of 1 mM to 20 mM, more preferably 5 mM to 10 mM.

Preferably, the nucleic acid lysis buffer consists of the components defined as above.

Preferably, the second solution comprises a second buffer; those skilled in the art will appreciate that a pH regulator may be added to adjust a pH value of the second buffer to 8 to 12, preferably 10 to 11. Preferably, the second buffer is selected from the group consisting of a DPBS buffer, a Tris buffer, a glycine-sodium hydroxide buffer, and a disodium hydrogen phosphate-sodium hydroxide buffer. Preferably, the Tris buffer, the glycine-sodium hydroxide buffer, and the disodium hydrogen phosphate-sodium hydroxide buffer each have a concentration of 1 mM to 30 mM, preferably 5 mM to 20 mM;
preferably, the third solution comprises a third buffer; those skilled in the art will appreciate that a pH regulator may be added to adjust a pH value of the third buffer to 3 to 6, preferably 5 to 6. Preferably, the third buffer is selected from the group consisting of a Tris buffer, an MES buffer, an acetic acid-sodium acetate buffer, and a citric acid-sodium citrate buffer. Preferably, the third buffer has a concentration of 1 mM to 20 mM, more preferably 5 mM to 15 mM; preferably, the third buffer further includes the metal chelator; preferably, the metal chelator includes EDTA and/or a salt of EDTA including disodium EDTA, tetrasodium EDTA, and calcium disodium EDTA; preferably, the metal chelator has a concentration of 0.5 mM to 10 mM, more preferably 1 mM to 5 mM.

In the present disclosure, the nucleic acid extraction element exists alone or is fixed on a rod-shaped support. Those skilled in the art will appreciate that the nucleic acid extraction element (including the nucleic acid extraction element fixed on the rod-shaped support) can be placed in the first solution or exist independently, and then placed in the first solution before, at the same time, or after the sample to be tested is placed in the first solution.

Preferably, the nucleic acid extraction element is arranged at or wrapped on one end of a rod-shaped support. For example, fibers (such as monofilament fibers) are fixed to one end of the rod-shaped support by winding and/or welding (such as melting by heating); alternatively, fibers (such as monofilament fibers) are first woven, needle-punched, or wound into a single-layer or multi-layer fabric, and then fixed to one end of the rod-shaped support by winding and/or welding (such as melting by heating). Preferably, the nucleic acid extraction element has a length of 1 cm to 2 cm in a direction along the rod-shaped support; and preferably, the fibers have a diameter of 0.05 mm to 0.5 mm, preferably 0.1 mm to 0.3 mm.

Preferably, the other end of the rod-shaped support is fixed inside a sealing element matched with the first container and the third container; in some embodiments of the present disclosure, the first container and the third container each are tubular, and the sealing element is a tube cap. Those skilled in the art will appreciate that the rod-shaped support may be fixed to an interior of the sealing element matched with the first container and the third container by methods known in the art, such as welding or threading.

Preferably, the rod-shaped support has a breakable point at one end close to the nucleic acid extraction element.

Preferably, the kit or set further includes a fourth container for packaging the nucleic acid extraction element. Preferably, the fourth container is matched with the sealing element to which the rod-shaped support is fixed; preferably, the fourth container is tubular; and preferably, the fourth container has no other contents and is only used for packaging the nucleic acid extraction element.

Preferably, the nucleic acid extraction element is placed in the first solution.

Preferably, the first solution has a volume of 1 mL to 10 mL, more preferably 1.5 mL to 3 mL;
preferably, the second solution has a volume of 100 μL to 500 μL, more preferably 150 μL to 350 μL;
preferably, the third solution has a volume of 1 mL to 10 mL, more preferably 2 mL to 4 mL.

Those skilled in the art will appreciate that the kit or set further includes a sampling element. In some embodiments, the sampling element is a throat swab. Of course, those skilled in the art will appreciate that the rod-shaped support fixed with the nucleic acid extraction element can also be used as the sampling element, such as a throat swab.

The present disclosure also provides use of polyacrylate, polyamide, polyester, polyurethane, or PLA in nucleic acid extraction.

The present disclosure also provides use of polyacrylate, polyamide, polyester, polyurethane, or PLA in preparation of a kit or a set for nucleic acid extraction or nucleic acid detection; where preferably, the use refers to preparing the nucleic acid extraction element.

In the present disclosure, sample collection and nucleic acid extraction can be conducted immediately, sequentially, or simultaneously, thereby making full use of duration consumed during sample transportation, and achieving nucleic acid purification within 4 min after the sample arrives at the laboratory. Moreover, a nucleic acid yield is comparable to that of the magnetic bead method currently on the market.

### BRIEF DESCRIPTION OF THE DRAWINGS

FIG. 1 shows a schematic diagram of a process of the nucleic acid extraction using a magnetic bead method; and

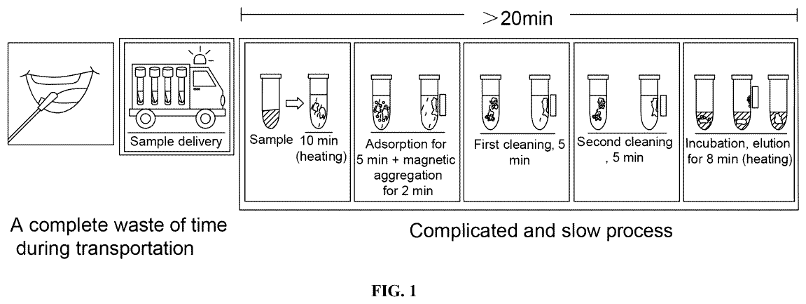

FIG. 2 shows a schematic diagram of a process of conducting nucleic acid extraction using the nucleic acid extraction element in the present disclosure.

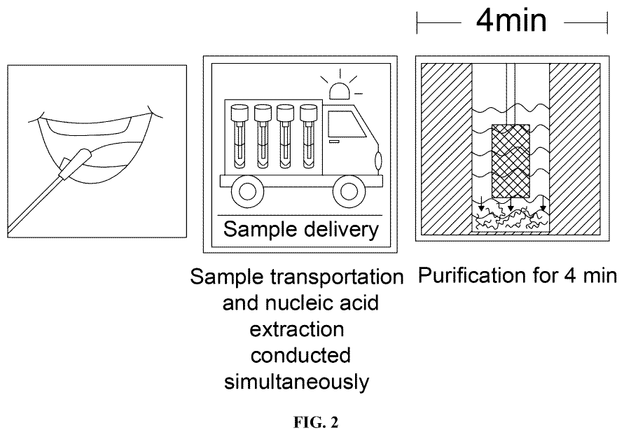

### DETAILED DESCRIPTION OF THE EMBODIMENTS

The present disclosure will be further described below with reference to specific examples. It should be understood that these embodiments are only intended to describe the present disclosure, rather than to limit the scope of the present disclosure. In addition, it should be understood that various changes and modifications may be made to the present disclosure by a person skilled in the art after reading the contents described in the present disclosure, and these equivalents also fall within the scope defined by the present disclosure.

In the examples, unless otherwise specified, the experimental methods used are conventional, and the materials and reagents used are commercially available.

In the description of the present disclosure, it should be noted that the terms “first”, “second”, “third”, “fourth”, and “fifth” in the description of the present disclosure are only used for description purpose and cannot be understood to indicate or imply relative importance.

The term “optionally” means that a feature may be present or absent, and also means that the feature must be present, but a specific selection is optional.

### Example 1

PET fiber (Nantong NTEC Monofilament Technology Co., Ltd., diameter 0.15 mm, D0150) and PLA fiber (Nantong NTEC Monofilament Technology Co., Ltd., diameter 0.25 mm, 0501) were used as warp and weft, respectively, to weave a mesh fabric with a width of about 2 cm. The mesh fabric was cut into a rectangle of about 5 cm in length, wrapped and welded to one end of an ABS rod of about 2 cm in length by heating melting, where the rod had a breakable point at about 3 cm from a top of the one end; and the other end of the rod was fixed to the interior of a tube cap that could be matched with a sampling tube and a cleaning tube by welding or threading. The nucleic acid extraction element made of the PET fiber and the PLA fiber in this example had a volume of about 0.4 cm³ and a surface area of about 16 cm².

A lysis buffer, a cleaning solution, and a purification solution were prepared:

A 10 mM MES buffer containing 0.6 M arginine, 0.5 M NaCl, and 5 mM EDTA was prepared, and adjusted to a pH value of 3 with a 1 M sodium hydroxide aqueous solution and/or a 1 M hydrochloric acid aqueous solution to obtain the lysis buffer, which was dispensed into sampling tubes, 2 mL per tube.

A 5 mM Tris buffer containing 3 mM disodium EDTA was prepared, and adjusted to a pH value of 3 with a 1 M sodium hydroxide aqueous solution and/or a 1 M hydrochloric acid aqueous solution to obtain the cleaning solution, which was dispensed into cleaning tubes, 2 mL per tube.

A DPBS buffer was prepared, and adjusted to a pH value of 9 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. An H1N1 virus solution was subjected to gradient dilution to a concentration of 5×10³ copies/mL.

2. 222 μL of H1N1 virus solution was added into a traditional inactivated sampling tube (Yocon Biology Technology Company, MT0501-7-3) containing 2 mL of the sampling solution, such that the H1N1 virus had a final concentration of 5×10² copies/mL. Similarly, 222 L of virus solution was added into the sampling tube prepared in this example, such that the H1N1 virus had a final concentration of 5×10² copies/mL, and then the sampling tube was covered using a tube cap with the nucleic acid extraction element prepared in this example, such that the nucleic acid extraction element was completely immersed in a mixture of the virus solution and the lysis buffer in the sampling tube.

3. The traditional inactivated sampling tube was allowed to stand for 0.5 h, and nucleic acid was extracted using a magnetic bead method-based nucleic acid extraction kit (Yocon Biology Technology Company, MK0102-100) according to instructions, and then fluorescence quantitative PCR was repeated 3 times to obtain results Ct-1, Ct-2, and Ct-3, and an average was taken. The results are shown in Table 1.

4. The sampling tube of the present disclosure was allowed to stand for 0.5 h, the tube cap with the nucleic acid extraction element was placed on the cleaning tube such that the nucleic acid extraction element was completely immersed in the cleaning solution in the cleaning tube, and then the cleaning tube was shaken vertically for about 30 s after screwing the tube cap.

5. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, and then fluorescence quantitative PCR was repeated 3 times to obtain results Ct-1, Ct-2 and Ct-3, and an average was taken. The results are shown in Table 1.

TABLE 1
Comparison of PCR results after nucleic acid extraction by
method of the present disclosure and magnetic bead method

| | The present disclosure | Magnetic bead method | P value |
|---|---|---|---|
| Ct-1 | 32.27 | 33.04 | 0.618 |
| Ct-2 | 32.78 | 32.28 | |
| Ct-3 | 32.03 | 32.30 | |
| Ct average | 32.36 | 32.54 | |

This indicated that there was no significant difference in the PCR results of nucleic acid extracted by the method of the present disclosure compared with the magnetic bead method. However, the method of the present disclosure was simple to operate and time-saving, and the cleaning and purification of nucleic acid only took about 4 min; while the extraction, cleaning, and purification of the magnetic bead method took about 20 min.

### Example 2

One end of 200 polyurethane fibers (Nantong NTEC Monofilament Technology Co., Ltd., diameter 0.2 mm, J0200) with a length of about 1 cm were melted and welded to the top of one end of the ABS rod within about 1.2 cm, where the rod had a breakable point at about 3 cm from a top of the one end; and the other end of the rod was fixed to the interior of a tube cap that could be matched with a sampling tube and a cleaning tube by welding or threading. The nucleic acid extraction element made of the polyurethane fibers in this example had a volume of about 0.5 cm³ and a surface area of about 14 cm².

A lysis buffer, a cleaning solution, and a purification solution were prepared:

A 15 mM MES buffer containing 0.8 M arginine, 1 M NaCl, and 8 mM disodium EDTA was prepared, and adjusted to a pH value of 4 with a 1 M sodium hydroxide aqueous solution and/or a 1 M hydrochloric acid aqueous solution to obtain the lysis buffer, which was dispensed into sampling tubes, 2 mL per tube.

A 10 mM Tris buffer containing 1 mM disodium EDTA was prepared, and adjusted to a pH value of 4 with a 1 M sodium hydroxide aqueous solution and/or a 1 M hydrochloric acid aqueous solution to obtain the cleaning solution, which was dispensed into cleaning tubes, 2 mL per tube.

A DPBS buffer was prepared, and adjusted to a pH value of 11 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. An H1N1 virus solution was subjected to gradient dilution to a concentration of 5×10³ copies/mL.

2. 222 μL of H1N1 virus solution was added into a traditional inactivated sampling tube (Yocon Biology Technology Company, MT0501-7-3) containing 2 mL of the sampling solution, such that the H1N1 virus had a final concentration of 5×10² copies/mL. Similarly, 222 μL of H1N1 virus solution was added into the sampling tube prepared in this example, such that the H1N1 virus had a final concentration of 5×10² copies/mL, and then the sampling tube was covered using a tube cap with the nucleic acid extraction element prepared in this example, such that the nucleic acid extraction element was completely immersed in a mixture of the virus solution and the lysis buffer in the sampling tube.

3. The traditional inactivated sampling tube was allowed to stand for 0.5 h, and nucleic acid was extracted using a magnetic bead method-based nucleic acid extraction kit (Yocon Biology Technology Company, MK0102-100) according to instructions, and then fluorescence quantitative PCR was repeated 3 times to obtain results Ct-1, Ct-2, and Ct-3, and an average was taken. The results are shown in Table 2.

4. The sampling tube of the present disclosure was allowed to stand for 0.5 h, the tube cap with the nucleic acid extraction element was placed on the cleaning tube such that the nucleic acid extraction element was completely immersed in the cleaning solution in the cleaning tube, and then the cleaning tube was shaken vertically for about 30 s after screwing the tube cap.

5. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, and then fluorescence quantitative PCR was repeated 3 times to obtain results Ct-1, Ct-2 and Ct-3, and an average was taken. The results are shown in Table 2.

TABLE 2
Comparison of PCR results after nucleic acid extraction by
method of the present disclosure and magnetic bead method

| | The present disclosure | Magnetic bead method | P value |
|---|---|---|---|
| Ct-1 | 32.94 | 32.44 | 0.612 |
| Ct-2 | 32.36 | 31.80 | |
| Ct-3 | 33.14 | 33.36 | |
| Ct average | 32.81 | 32.53 | |

### Example 3

PLA fiber (Nantong NTEC Monofilament Technology Co., Ltd., diameter 0.25 mm, 0501) was woven into a mesh fabric with a width of about 2 cm. The mesh fabric was cut into a rectangle of about 5 cm in length, wrapped and welded to one end of an ABS rod of about 2 cm in length by heating melting, where the rod had a breakable point at about 3 cm from a top of the one end; and the other end of the rod was fixed to the interior of a tube cap that could be matched with a sampling tube and a cleaning tube by welding or threading. The nucleic acid extraction element made of the PLA fiber in this example had a volume of about 0.5 cm³ and a surface area of about 13 cm².

A lysis buffer, a cleaning solution, and a purification solution were prepared:

A 25 mM acetic acid-sodium acetate buffer containing 0.6 M arginine, 1.2 M NaCl, and 5 mM disodium EDTA was prepared, and adjusted to a pH value of 6 with a 1 M sodium hydroxide aqueous solution and/or a 1 M hydrochloric acid aqueous solution to obtain the lysis buffer, which was dispensed into sampling tubes, 2 mL per tube.

A 15 mM Tris buffer containing 5 mM disodium EDTA was prepared, and adjusted to a pH value of 6 with a 1 M sodium hydroxide aqueous solution and/or a 1 M hydrochloric acid aqueous solution to obtain the cleaning solution, which was dispensed into cleaning tubes, 2 mL per tube.

A 5 mM Tris buffer was prepared, and adjusted to a pH value of 8 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. An H1N1 virus solution was subjected to gradient dilution to a concentration of 5×10³ copies/mL.

2. 222 μL of H1N1 virus solution was added into a traditional inactivated sampling tube (Yocon Biology Technology Company, MT0501-7-3) containing 2 mL of the sampling solution, such that the H1N1 virus had a final concentration of 5×10² copies/mL. Similarly, 222 μL of virus solution was added into the sampling tube prepared in this example, such that the H1N1 virus had a final concentration of 5×10² copies/mL, and then the sampling tube was covered using a tube cap with the nucleic acid extraction element prepared in this example, such that the nucleic acid extraction element was completely immersed in a mixture of the virus solution and the lysis buffer in the sampling tube.

3. The traditional inactivated sampling tube was allowed to stand for 0.5 h, and nucleic acid was extracted using a magnetic bead method-based nucleic acid extraction kit (Yocon Biology Technology Company, MK0102-100) according to instructions, and then fluorescence quantitative PCR was repeated 3 times to obtain results Ct-1, Ct-2, and Ct-3, and an average was taken. The results are shown in Table 3.

4. The sampling tube of the present disclosure was allowed to stand for 0.5 h, the tube cap with the nucleic acid extraction element was placed on the cleaning tube such that the nucleic acid extraction element was completely immersed in the cleaning solution in the cleaning tube, and then the cleaning tube was shaken vertically for about 30 s after screwing the tube cap.

5. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, and then fluorescence quantitative PCR was repeated 3 times to obtain results Ct-1, Ct-2 and Ct-3, and an average was taken. The results are shown in Table 3.

TABLE 3
Comparison of PCR results after nucleic acid extraction by
method of the present disclosure and magnetic bead method

| | The present disclosure | Magnetic bead method | P value |
|---|---|---|---|
| Ct-1 | 32.38 | 33.44 | 0.057 |
| Ct-2 | 32.64 | 33.32 | |
| Ct-3 | 33.20 | 33.40 | |
| Ct average | 32.74 | 33.39 | |

### Example 4

PMMA fiber (Shenzhen Chuangli Fiber Optic Materials Co., Ltd., diameter 0.2 mm, CL-250) was woven into a mesh fabric with a width of about 2 cm. The mesh fabric was cut into a rectangle of about 5 cm in length, wrapped and welded to one end of an ABS rod of about 2 cm in length by heating melting, where the rod had a breakable point at about 3 cm from a top of the one end; and the other end of the rod was fixed to the interior of a tube cap that could be matched with a sampling tube and a cleaning tube by welding or threading. The nucleic acid extraction element made of the PMMA fiber in this example had a volume of about 0.6 cm³ and a surface area of about 16 cm².

A lysis buffer, a cleaning solution, and a purification solution were prepared:

A 10 mM acetic acid-sodium acetate buffer containing 1 M arginine, 0.5 M KCl, and 10 mM EDTA was prepared, and adjusted to a pH value of 6 with a 1 M sodium hydroxide aqueous solution and/or a 1 M hydrochloric acid aqueous solution to obtain the lysis buffer, which was dispensed into sampling tubes, 2 mL per tube.

A 5 mM MES buffer containing 5 mM disodium EDTA was prepared, and adjusted to a pH value of 4 with a 1 M sodium hydroxide aqueous solution and/or a 1 M hydrochloric acid aqueous solution to obtain the cleaning solution, which was dispensed into cleaning tubes, 2 mL per tube.

A 10 mM Tris buffer was prepared, and adjusted to a pH value of 9 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. An H1N1 virus solution was subjected to gradient dilution to a concentration of 5×10³ copies/mL.

2. 222 μL of H1N1 virus solution was added into a traditional inactivated sampling tube (Yocon Biology Technology Company, MT0501-7-3) containing 2 mL of the sampling solution, such that the H1N1 virus had a final concentration of 5×10² copies/mL. Similarly, 222 μL of virus solution was added into the sampling tube prepared in this example, such that the H1N1 virus had a final concentration of 5×10² copies/mL, and then the sampling tube was covered using a tube cap with the nucleic acid extraction element prepared in this example, such that the nucleic acid extraction element was completely immersed in a mixture of the virus solution and the lysis buffer in the sampling tube.

3. The traditional inactivated sampling tube was allowed to stand for 0.5 h, and nucleic acid was extracted using a magnetic bead method-based nucleic acid extraction kit (Yocon Biology Technology Company, MK0102-100) according to instructions, and then fluorescence quantitative PCR was repeated 3 times to obtain results Ct-1, Ct-2, and Ct-3, and an average was taken. The results are shown in Table 4.

4. The sampling tube of the present disclosure was allowed to stand for 0.5 h, the tube cap with the nucleic acid extraction element was placed on the cleaning tube such that the nucleic acid extraction element was completely immersed in the cleaning solution in the cleaning tube, and then the cleaning tube was shaken vertically for about 30 s after screwing the tube cap.

5. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, and then fluorescence quantitative PCR was repeated 3 times to obtain results Ct-1, Ct-2 and Ct-3, and an average was taken. The results are shown in Table 4.

TABLE 4
Comparison of PCR results after nucleic acid extraction by
method of the present disclosure and magnetic bead method

| | The present disclosure | Magnetic bead method | P value |
|---|---|---|---|
| Ct-1 | 32.52 | 32.13 | 0.547 |
| Ct-2 | 32.62 | 32.93 | |
| Ct-3 | 32.40 | 33.07 | |
| Ct average | 32.51 | 32.71 | |

### Example 5

PMMA fiber (Shenzhen Chuangli Fiber Optic Materials Co., Ltd., diameter 0.2 mm, CL-250) was woven into a mesh fabric with a width of about 2 cm. The mesh fabric was cut into a rectangle of about 5 cm in length, wrapped and welded to one end of an ABS rod of about 2 cm in length by heating melting, where the rod had a breakable point at about 3 cm from a top of the one end; and the other end of the rod was fixed to the interior of a tube cap that could be matched with a sampling tube and a cleaning tube by welding or threading. The nucleic acid extraction element made of the polyurethane fibers in this example had a volume of about 0.4 cm³ and a surface area of about 14 cm².

A lysis buffer, a cleaning solution, and a purification solution were prepared:

A 15 mM MES buffer containing 0.8 M arginine, 1.0 M KCl, and 8 mM EDTA was prepared, and adjusted to a pH value of 3 with a 1 M sodium hydroxide aqueous solution and/or a 1 M hydrochloric acid aqueous solution to obtain the lysis buffer, which was dispensed into sampling tubes, 2 mL per tube.

A 10 mM MES buffer containing 1 mM disodium EDTA was prepared, and adjusted to a pH value of 6 with a 1 M sodium hydroxide aqueous solution and/or a 1 M hydrochloric acid aqueous solution to obtain the cleaning solution, which was dispensed into cleaning tubes, 2 mL per tube.

A 15 mM Tris buffer was prepared, and adjusted to a pH value of 10 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. MDCK cells were cultured until their confluence reached 90%, digested with EDTA trypsin, collected into a centrifuge tube, centrifuged at 800 rpm for 5 min, a supernatant was discarded to obtain a cell pellet, 1 mL of PBS was added to resuspend the cell pellet, and an obtained cell suspension was counted, with a density of 1×10⁷ cells/mL.

2. 222 μL of cell suspension was added into the sampling tube prepared in this example, and then the sampling tube was covered using a tube cap with the nucleic acid extraction element prepared in this example, such that the nucleic acid extraction element was completely immersed in a mixture of the cell suspension and the lysis buffer in the sampling tube.

3. After shaking in a shaker at 150 rpm for 0.5 h at 37° C., the tube cap with the nucleic acid extraction element was placed on the cleaning tube such that the nucleic acid extraction element was completely immersed in the cleaning solution in the cleaning tube, and then the cleaning tube was shaken vertically for about 30 s after screwing the tube cap.

4. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, and then the nucleic acid content was detected using an ultra-micro UV spectrophotometer. The results are shown in Table 5.

222 μL of cell suspension was added into a conventional inactivated sampling tube (Yocon Biology Technology Company, MT0501-7-3) containing 2 mL of sampling solution, and allowed to stand for 0.5 h. The nucleic acid was extracted using a magnetic bead method-based nucleic acid extraction kit (Yocon Biology Technology Company, MK0102-100) according to instructions, and then the nucleic acid content was detected using an ultra-micro UV spectrophotometer. The results are shown in Table 5.

TABLE 5
Results of extracting cell nucleic acid in the present disclosure

| | Concentration of | |
|---|---|---|
| | nucleic acid | |
| | extracted in the | Concentration of nucleic acid extracted |
| Nucleic acid | present disclosure | in control magnetic bead method |
| DNA | 9.88 ng/μL | 9.55 ng/μL |
| RNA | 9.21 ng/μL | 9.76 ng/μL |

### Example 6

Polyamide fiber (PA66, Nantong NTEC Monofilament Technology Co., Ltd., diameter 0.25 mm,

J250D) was woven into a mesh fabric with a width of about 2 cm. The mesh fabric was cut into a rectangle of about 5 cm in length, wrapped and welded to one end of an ABS rod of about 2 cm in length by heating melting, where the rod had a breakable point at about 3 cm from a top of the one end; and the other end of the rod was fixed to the interior of a tube cap that could be matched with a sampling tube and a cleaning tube by welding or threading. The nucleic acid extraction element made of polyamide fiber in this example had a volume of about 0.4 cm³ and a surface area of about 16 cm².

A lysis buffer, a cleaning solution, and a purification solution were prepared:

A 25 mM acetic acid-sodium acetate buffer containing 0.5 M arginine, 1.2 M KCl, and 6 mM EDTA was prepared, and adjusted to a pH value of 4 with a 1 M sodium hydroxide aqueous solution and/or a 1 M hydrochloric acid aqueous solution to obtain the lysis buffer, which was dispensed into sampling tubes, 2 mL per tube.

A 15 mM MES buffer containing 5 mM disodium EDTA was prepared, and adjusted to a pH value of 5 with a 1 M sodium hydroxide aqueous solution and/or a 1 M hydrochloric acid aqueous solution to obtain the cleaning solution, which was dispensed into cleaning tubes, 2 mL per tube.

A 5 mM glycine-sodium hydroxide buffer was prepared, and adjusted to a pH value of 10 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. An H1N1 virus solution was subjected to gradient dilution to a concentration of 5×10³ copies/mL.

2. 222 μL of H1N1 virus solution was added into a traditional inactivated sampling tube (Yocon Biology Technology Company, MT0501-7-3) containing 2 mL of the sampling solution, such that the H1N1 virus had a final concentration of 5×10² copies/mL. Similarly, 222 μL of virus solution was added into the sampling tube prepared in this example, such that the H1N1 virus had a final concentration of 5×10² copies/mL, and then the sampling tube was covered using a tube cap with the nucleic acid extraction element prepared in this example, such that the nucleic acid extraction element was completely immersed in a mixture of the virus solution and the lysis buffer in the sampling tube.

3. The traditional inactivated sampling tube was allowed to stand for 0.5 h, and nucleic acid was extracted using a magnetic bead method-based nucleic acid extraction kit (Yocon Biology Technology Company, MK0102-100) according to instructions, and then fluorescence quantitative PCR was repeated 3 times to obtain results Ct-1, Ct-2, and Ct-3, and an average was taken. The results are shown in Table 6.

4. The sampling tube of the present disclosure was allowed to stand for 0.5 h, the tube cap with the nucleic acid extraction element was placed on the cleaning tube such that the nucleic acid extraction element was completely immersed in the cleaning solution in the cleaning tube, and then the cleaning tube was shaken vertically for about 30 s after screwing the tube cap.

5. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, and then fluorescence quantitative PCR was repeated 3 times to obtain results Ct-1, Ct-2 and Ct-3, and an average was taken. The results are shown in Table 6.

TABLE 6
Comparison of PCR results after nucleic acid extraction by
method of the present disclosure and magnetic bead method

| | The present disclosure | Magnetic bead method | P value |
|---|---|---|---|
| Ct-1 | 33.39 | 33.08 | 0.528 |
| Ct-2 | 32.99 | 33.79 | |
| Ct-3 | 33.07 | 33.12 | |
| Ct average | 33.15 | 33.33 | |

### Example 7

PET fiber (Nantong NTEC Monofilament Technology Co., Ltd., diameter 0.15 mm, D0150) and PLA fiber (Nantong NTEC Monofilament Technology Co., Ltd., diameter 0.25 mm, 0501) were used as warp and weft, respectively, to weave a mesh fabric with a width of about 2 cm. The mesh fabric was cut into a rectangle of about 5 cm in length, wrapped and welded to one end of an ABS rod of about 2 cm in length by heating melting, where the rod had a breakable point at about 3 cm from a top of the one end; and the other end of the rod was fixed to the interior of a tube cap that could be matched with a sampling tube and a cleaning tube by welding or threading. The nucleic acid extraction element made of the PET fiber and the PLA fiber in this example had a volume of about 0.4 cm³ and a surface area of about 16 cm².

A lysis buffer, a cleaning solution, and a purification solution were prepared:

A 10 mM MES buffer containing 0.5 M arginine, 0.5 M sodium citrate, and 6 mM EDTA was prepared, and adjusted to a pH value of 5 with a 1 M sodium hydroxide aqueous solution and/or a 1 M hydrochloric acid aqueous solution to obtain the lysis buffer, which was dispensed into sampling tubes, 2 mL per tube.

A 10 mM acetic acid-sodium acetate buffer containing 2 mM disodium EDTA was prepared, and adjusted to a pH value of 3 with a 1 M sodium hydroxide aqueous solution and/or a 1 M hydrochloric acid aqueous solution to obtain the cleaning solution, which was dispensed into cleaning tubes, 2 mL per tube.

A 15 mM glycine-sodium hydroxide buffer was prepared, and adjusted to a pH value of 11 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. A PEDV virus solution was subjected to gradient dilution to a concentration of 5×10³ copies/mL.

2. 222 μL of PEDV virus solution was added into the sampling tube prepared in this example, such that the PEDV virus had a final concentration of 5×10² copies/mL, and then the sampling tube was covered using a tube cap with the nucleic acid extraction element prepared in this example, such that the nucleic acid extraction element was completely immersed in a mixture of the virus solution and the lysis buffer in the sampling tube, and a total of 7 tubes were set up for replication.

3. One of the tubes was allowed to stand for 10 min, the tube cap with the nucleic acid extraction element was placed on the cleaning tube such that the nucleic acid extraction element was completely immersed in the cleaning solution in the cleaning tube, and then the cleaning tube was shaken vertically for about 30 s after screwing the tube cap.

4. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, to obtain purified nucleic acid, which was frozen at −80° C. and recorded as the nucleic acid extracted on day 1.

5. The remaining six tubes were allowed to stand for 2 days to 7 days and then steps 3 and 4 were repeated to obtain nucleic acids from 2 d to 7 d, respectively.

6. Fluorescence quantitative PCR was conducted 2 times on the nucleic acids from day 1 to day 7 to obtain results Ct-1 and Ct-2, an average was taken, and whether there were significant differences at different times was compared. The test results are shown in Table 7.

TABLE 7
Statistical results of Ct values detected at different time points

| Time | 10 min | day 2 | day 3 | day 4 | day 5 | day 6 | day 7 |
|---|---|---|---|---|---|---|---|
| Ct-1 | 32.57 | 33.79 | 33.35 | 32.58 | 33.47 | 33.13 | 33.07 |
| Ct-2 | 32.91 | 33.41 | 32.69 | 32.76 | 33.19 | 32.56 | 32.85 |
| Ct | 32.74 | 33.6 | 33.02 | 32.67 | 33.33 | 32.845 | 32.96 |
| average | | | | | | | |

It was seen that there was no significant difference in PCR detection of nucleic acids extracted by the method of the present disclosure when the extraction time was as short as 10 min and as long as 7 days, indicating that the method was extremely stable.

### Example 8

PLA fiber (Nantong NTEC Monofilament Technology Co., Ltd., diameter 0.25 mm, 0501) was woven into a mesh fabric with a width of about 2 cm. The mesh fabric was cut into a rectangle of about 5 cm in length, wrapped and welded to one end of an ABS rod of about 2 cm in length by heating melting, where the rod had a breakable point at about 3 cm from a top of the one end; and the other end of the rod was fixed to the interior of a tube cap that could be matched with a sampling tube and a cleaning tube by welding or threading. The nucleic acid extraction element made of the PLA fiber in this example had a volume of about 0.4 cm³ and a surface area of about 16 cm².

A lysis buffer, a cleaning solution, and a purification solution were prepared:

A 15 mM citric acid-sodium citrate buffer containing 0.7 M arginine, 1 M sodium citrate, and 8 mM disodium EDTA was prepared, and adjusted to a pH value of 2 with a 1 M sodium hydroxide aqueous solution and/or a 1 M hydrochloric acid aqueous solution to obtain the lysis buffer, which was dispensed into sampling tubes, 2 mL per tube.

A 15 mM acetic acid-sodium acetate buffer containing 5 mM disodium EDTA was prepared, and adjusted to a pH value of 5 with a 1 M sodium hydroxide aqueous solution and/or a 1 M hydrochloric acid aqueous solution to obtain the cleaning solution, which was dispensed into cleaning tubes, 2 mL per tube.

A 20 mM glycine-sodium hydroxide buffer was prepared, and adjusted to a pH value of 12 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. A PEDV virus solution was subjected to gradient dilution to a concentration of 5×10⁴ copies/mL.

2. 222 μL of PEDV virus solution was added into the sampling tube prepared in this example, such that the PEDV virus had a final concentration of 5×10² copies/mL, and then the sampling tube was covered using a tube cap with the nucleic acid extraction element prepared in this example, such that the nucleic acid extraction element was completely immersed in a mixture of the virus solution and the lysis buffer in the sampling tube, and a total of 4 tubes were set up for replication.

3. The 4 tubes were placed in environments of 4° C., 25° C., 37° C., and 60° C., and shaken at 150 rpm for 0.5 h.

4. After 0.5 h, the tube cap with the nucleic acid extraction element in each tube was placed on the cleaning tube such that the nucleic acid extraction element was completely immersed in the cleaning solution in the cleaning tube, and then the cleaning tube was shaken vertically for about 30 s after screwing the tube cap.

5. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, and then fluorescence quantitative PCR was repeated 2 times to obtain results Ct-1 and Ct-2, and an average was taken. The results are shown in Table 8.

TABLE 8
Statistics of nucleic acid detection results of each group
obtained by extraction at 4° C. to 60° C.

| Delivery temperature | ° C. | ° C. | 37° C. | 60° C. |
|---|---|---|---|---|
| Ct-1 | 30.45 | 30.07 | 29.63 | 29.69 |
| Ct-2 | 29.61 | 29.85 | 29.27 | 29.27 |
| Ct average | 30.03 | 29.96 | 29.45 | 29.48 |

It was seen that there was no significant difference in the PCR detection of nucleic acids extracted by the method of the present disclosure when the extraction temperature was as low as 4° C. and as high as 60° C., indicating that the method was extremely stable.

### Example 9

PMMA fiber (Shenzhen Chuangli Fiber Optic Materials Co., Ltd., diameter 0.2 mm, CL-250) and PLA fiber (Nantong NTEC Monofilament Technology Co., Ltd., diameter 0.25 mm, 0501) were used as warp and weft, respectively, to weave a mesh fabric with a width of about 2 cm. The mesh fabric was cut into a rectangle of about 5 cm in length, wrapped and welded to one end of an ABS rod of about 2 cm in length by heating melting, where the rod had a breakable point at about 3 cm from a top of the one end; and the other end of the rod was fixed to the interior of a tube cap that could be matched with a sampling tube and a cleaning tube by welding or threading. The nucleic acid extraction element made of the PMMA fiber and the PLA fiber in this example had a volume of about 0.5 cm³ and a surface area of about 13 cm².

A lysis buffer, a cleaning solution, and a purification solution were prepared:

A 25 mM citric acid-sodium citrate buffer containing 0.7 M arginine, 1.2 M sodium citrate, and 10 mM EDTA was prepared, and adjusted to a pH value of 6 with a 1 M sodium hydroxide aqueous solution and/or a 1 M hydrochloric acid aqueous solution to obtain the lysis buffer, which was dispensed into sampling tubes, 2 mL per tube.

A 15 mM citric acid-sodium citrate buffer containing 5 mM disodium EDTA was prepared, and adjusted to a pH value of 6 with a 1 M sodium hydroxide aqueous solution and/or a 1 M hydrochloric acid aqueous solution to obtain the cleaning solution, which was dispensed into cleaning tubes, 2 mL per tube.

A 5 mM sodium hydrogen phosphate-sodium hydroxide buffer was prepared, and adjusted to a pH value of 10 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. An H1N1 virus solution was subjected to gradient dilution to a concentration of 5×10³ copies/mL.

2. 2 parts of 222 μL of H1N1 virus solution were added into traditional inactivated sampling tubes (Yocon Biology Technology Company, MT0501-7-3 and MT0501-2, the lysis buffer contained guanidine salt) containing 2 mL of the sampling solution, such that the H1N1 virus had a final concentration of 5×10² copies/mL. Similarly, 222 μL of H1N1 virus solution was added into the sampling tube prepared in this example, such that the H1N1 virus had a final concentration of 5×10² copies/mL, and then the above three sampling tubes were covered using a tube cap with the nucleic acid extraction element prepared in this example, such that the nucleic acid extraction element was completely immersed in a mixture of the virus solution and the lysis buffer in the sampling tube.

3. The three sampling tubes were allowed to stand for 0.5 h, the tube cap with the nucleic acid extraction element was placed on the cleaning tube such that the nucleic acid extraction element was completely immersed in the cleaning solution in the cleaning tube, and then the cleaning tube was shaken vertically for about 30 s after screwing the tube cap.

4. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, and then fluorescence quantitative PCR was repeated 3 times to obtain results Ct-1, Ct-2 and Ct-3, and an average was taken. The results are shown in Table 9.

TABLE 9
Comparison of PCR results after nucleic acid extraction by method
of the present disclosure and replacing different lysis buffers

| | | Commercially | Commercially |
|---|---|---|---|
| | | available | available |
| | In this example | lysis | lysis |
| | Lysis buffer | buffer (1) | buffer (2) |
| Ct-1 | 31.49 | 32.72 | 32.98 |
| Ct-2 | 31.49 | 33.36 | 32.71 |
| Ct average | 31.49 | 33.04 | 32.85 |

It was seen that the method of the present disclosure could effectively detect nucleic acids using various lysis buffers, and the effect of the lysis buffer without guanidine salt was better than that of the lysis buffer containing guanidine salt.

### Example 10

Polyamide fiber (PA66, Nantong NTEC Monofilament Technology Co., Ltd., diameter 0.25 mm, J250D) and PLA fiber (Nantong NTEC Monofilament Technology Co., Ltd., diameter 0.25 mm, 0501) were used as warp and weft, respectively, to weave a mesh fabric with a width of about 2 cm. The mesh fabric was cut into a rectangle of about 5 cm in length, wrapped and welded to one end of an ABS rod of about 2 cm in length by heating melting, where the rod had a breakable point at about 3 cm from a top of the one end; and the other end of the rod was fixed to the interior of a tube cap that could be matched with a sampling tube and a cleaning tube by welding or threading. The nucleic acid extraction element made of the polyamide fiber and the PLA fiber in this example had a volume of about 0.6 cm³ and a surface area of about 16 cm².

A lysis buffer, a cleaning solution, and a purification solution were prepared:

A 25 mM acetic acid-sodium acetate buffer containing 0.8 M arginine, 0.5 M NaCl, and 5 mM EDTA was prepared, and adjusted to a pH value of 4 with a 1 M sodium hydroxide aqueous solution and/or a 1 M hydrochloric acid aqueous solution to obtain the lysis buffer, which was dispensed into sampling tubes, 2 mL per tube.

A 5 mM citric acid-sodium citrate buffer containing 2 mM disodium EDTA was prepared, and adjusted to a pH value of 4 with a 1 M sodium hydroxide aqueous solution and/or a 1 M hydrochloric acid aqueous solution to obtain the cleaning solution, which was dispensed into cleaning tubes, 2 mL per tube.

A 5 mM sodium hydrogen phosphate-sodium hydroxide buffer was prepared, and adjusted to a pH value of 11 with a 1 M sodium hydroxide aqueous solution and/or a 1 M hydrochloric acid aqueous solution to obtain the purification solution, which was dispensed into purification tubes, 300 μL per tube.

Method for extraction and detection of nucleic acid:

1. A PEDV virus solution was subjected to gradient dilution to a concentration of 5×10³ copies/mL.

2. 2 parts of 222 μL of PEDV virus solution were added into traditional inactivated sampling tubes (Yocon Biology Technology Company, MT0501-7-3 and MT0501-2, the lysis buffer contained guanidine salt) containing 2 mL of the sampling solution, such that the PEDV virus had a final concentration of 5×10² copies/mL. Similarly, 222 μL of PEDV virus solution was added into the sampling tube prepared in this example, such that the PEDV virus had a final concentration of 5×10² copies/mL, and then the above three sampling tubes were covered using a tube cap with the nucleic acid extraction element prepared in this example, such that the nucleic acid extraction element was completely immersed in a mixture of the virus solution and the lysis buffer in the sampling tube.

3. The three sampling tubes were allowed to stand for 0.5 h, the tube cap with the nucleic acid extraction element was placed on the cleaning tube such that the nucleic acid extraction element was completely immersed in the cleaning solution in the cleaning tube, and then the cleaning tube was shaken vertically for about 30 s after screwing the tube cap.

4. The tube cap with the nucleic acid extraction element was unscrewed, the rod was broken at the breakable point to fold one end of the nucleic acid extraction element into the purification tube, such that the nucleic acid extraction element was completely immersed in the purification solution of the purification tube. After screwing the tube cap, the purification tube was vortex-oscillated for about 3 min, and then fluorescence quantitative PCR was repeated 3 times to obtain results Ct-1, Ct-2 and Ct-3, and an average was taken. The results are shown in Table 10.

TABLE 10
Comparison of PCR results after nucleic acid extraction by method
of the present disclosure and replacing different lysis buffers

| | | Commercially | Commercially |
|---|---|---|---|
| | | available | available |
| | In this example | lysis | lysis |
| | Lysis buffer | buffer (1) | buffer (2) |
| Ct-1 | 30.38 | 30.61 | 30.44 |
| Ct-2 | 30.26 | 30.79 | 31.13 |
| Ct average | 30.32 | 30.70 | 30.87 |

The foregoing illustrates the implementation of the present disclosure. However, the present disclosure is not limited to the aforementioned implementation. Any modifications, equivalent substitutions, improvements, etc. made within the spirit and scope of the present disclosure should be included within the protection scope of the present disclosure.

1. A nucleic acid extraction element, comprising one or more of polyacrylate, polyamide, polyester, polyurethane, and polylactic acid (PLA), wherein the extraction element adsorbs nucleic acid in a first solution with a pH value of 2 to 6, and releases the nucleic acid in a second solution with a pH value of 8 to 12.

2. A method for extracting or detecting a nucleic acid, comprising the following steps: placing a sample to be tested in a first solution with a pH value of 2 to 6 such that a nucleic acid extraction element adsorbs the nucleic acid; and allowing the nucleic acid extraction element to release the nucleic acid in a second solution with a pH value of 8 to 12;
wherein the nucleic acid extraction element comprises one or more of polyacrylate, polyamide, polyester, polyurethane, and polylactic acid (PLA), and wherein the extraction element adsorbs nucleic acid in a first solution with a pH value of 2 to 6, and releases the nucleic acid in a second solution with a pH value of 8 to 12.

3. A kit or set for nucleic acid extraction or detection, comprising the nucleic acid extraction element according to claim 1.

4. The kit or set according to claim 3, wherein the nucleic acid extraction element is arranged at or wrapped on one end of a rod-shaped support.

5. The kit or set according to claim 3, wherein the nucleic acid extraction element is placed in the first solution.

6. The kit or set according to claim 3, wherein the first solution has a volume of 1 mL to 10 mL; the second solution has a volume of 100 μL to 500 μL; and the third solution has a volume of 1 mL to 10 mL.

7. The nucleic acid extraction element according to claim 1, wherein the nucleic acid comprises DNA and/or RNA and/or a fragment thereof.

8. The nucleic acid extraction element according to claim 1, the polyacrylate is polymethyl methacrylate (PMMA).

9. The nucleic acid extraction element according to claim 1, the polyester is polyethylene terephthalate (PET).

10. The nucleic acid extraction element according to claim 1, the nucleic acid extraction element has a volume of (0.1-2) cm³.

11. The nucleic acid extraction element according to claim 1, the nucleic acid extraction element has a surface area of not less than 5 cm².

12. The nucleic acid extraction element according to claim 1, the nucleic acid extraction element is arranged at or wrapped on one end of a rod-shaped support.

13. The nucleic acid extraction element according to claim 1, the nucleic acid extraction element has a length of 1 cm to 2 cm in a direction along the rod-shaped support.

14. The nucleic acid extraction element according to claim 1, the rod-shaped support has a breakable point at one end close to the nucleic acid extraction element.

15. The nucleic acid extraction or detection kit or set according to claim 3, wherein the kit or the set further comprises a first solution with a pH value of 2 to 6 that is placed in a first container.

16. The nucleic acid extraction or detection kit or set according to claim 3, wherein the kit or the set further comprises a second solution with a pH value of 8 to 12 that is placed in a second container.

17. The nucleic acid extraction or detection kit or set according to claim 3, wherein the kit or the set further comprises a third solution with a pH value of 3 to 6 that is placed in a third container.
